(19) United States
(12) Patent Application Publication
(10) Pub. No.: US 2025/0064567 A1
(43) Pub. Date: Feb. 27, 2025

(54) PATIENT SPECIFIC TOTAL TALUS FOR TOTAL ANKLE ARTHROPLASTY

(71) Applicant: Arthrex, Inc., Naples, FL (US)

(72) Inventors: Adam N. Garlock, Naples, FL (US); Robert Gorsline, Columbus, OH (US); Braham Dhillon, Naples, FL (US); Brian Dorn, Naples, FL (US); Andrea Matuska, Naples, FL (US)

(21) Appl. No.: 18/845,568

(22) PCT Filed: Mar. 21, 2023
(86) PCT No.: PCT/US2023/064755

§ 371 Date: Sep. 10, 2024

Related U.S. Application Data
(60) Provisional application No. 63/337,556, filed on May 2, 2022.
(60) Provisional application No. 63/322,178, filed on Mar. 21, 2022.

(51) Int. Cl. A61F 2/00; A61F 2/42

(52) U.S. Cl. CPC A61F 2/00; A61F 2002/4207; A61F 2310/00023

(57) ABSTRACT

A prosthetic talus comprising: a base having a top surface and a bottom surface; and an articulating component having a top surface and a bottom surface, wherein the bottom surface of the articulating component is removably coupled to the top surface of the base, wherein the bottom surface of the articulating component includes a protrusion, and wherein the top surface of the base includes a recess configured to receive the protrusion to thereby removably couple the articulating component to the base, wherein the articulating component includes a sidewall positioned between the top surface and the bottom surface, and wherein the sidewall includes a plurality of holes.

### CROSS-REFERENCE TO RELATED APPLICATIONS

This application claims the benefit of priority to (i) U.S. Provisional Application No. 63/322,178 entitled “Patient Specific Total Talus for Total Ankle Arthroplasty,” filed on Mar. 21, 2022, and (ii) U.S. Provisional Application No. 63/337,556 entitled “Prosthetic Implant with a Zinc-Strontium Alloy for Stem Cell Stimulation,” filed on May 2, 2022, the contents of each of which are hereby incorporated by reference in their entirety.

### BACKGROUND

Avascular necrosis (AVN) of the ankle typically occurs when the talus is damaged from a fracture that causes the talus to break down, resulting in severe pain and arthritis of the ankle. A treatment for AVN can be an ankle fusion (such as a tibio-talo-calcaneal (TTC) fusion). More recently, a total talus replacement has been used to address the specific challenges of AVN of the ankle. However, if a total talus replacement fails, the revision surgeries may present complications for both the surgeon and the patient. Disclosed herein is a prosthetic talus that can address complications associated with revision surgeries.

### SUMMARY

The disclosure herein includes a prosthetic talus for a prosthetic ankle. The prosthetic talus described herein can address complications associated with a failed TAR, a failed total talus replacement, a failed ankle fusion, or an AVN talus.

The prosthetic talus described herein provides a modular total talus that allows for a defined section of the total talus to be disconnected from a base of an implant. One example would allow an articulating component to be removable (e.g., via a morse taper connection as a non-limiting example) which would allow a surgeon to replace just a proximal component if further articular damage occurred on the tibia. This would also allow the surgeon to convert a total talus replacement to a total ankle replacement by swapping the proximal component for a new articulating component that matched the mating geometry of a poly insert of varying total ankle implants. Such a modular system is especially useful when implanting a total talus that is also fusing the subtalar joint or talonavicular joints, since these fusions would not need to be disrupted to remove the entire total talus implant. Furthermore, a prosthetic talus described herein may include suture eyelets or pre-tapped holes for accepting bone anchors, which would allow the surgeon to reattach the surrounding ankle ligaments to the implant. Providing such fixation methods for dermal or synthetic allografts could delay or inhibit the damage caused by the metal articulating component of the prosthetic talus.

Thus, in one aspect, the present disclosure provides a prosthetic talus including a base having a top surface and a bottom surface, and an articulating component having a top surface and a bottom surface. The bottom surface of the articulating component is removably coupled to the top surface of the base.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is an exploded perspective view of an example prosthetic talus of a prosthetic ankle.

FIG. 2 is an assembled perspective view of the example prosthetic talus of FIG. 1.

FIG. 3 is an anteroposterior view of an example prosthetic talus of a prosthetic ankle.

FIG. 4 is an exploded perspective view of the example prosthetic talus of FIG. 3.

FIG. 5 is an assembled perspective view of the example prosthetic talus of FIG. 3.

FIG. 6 is side cross-sectional view of the example articulating component of the prosthetic talus of FIG. 1 illustrating a lattice structure.

FIG. 7 is an example articulating component of a prosthetic talus illustrating a shell structure with an internal honeycomb structure.

### DETAILED DESCRIPTION

Total talus replacement surgery has been used to address various maladies in the ankle (e.g., avascular necrosis (AVN) of the ankle as a non-limiting example). However, if a total talus replacement fails, the revision surgeries may present complications for both the surgeon and the patient. The prosthetic talus described herein can address complications associated with a failed TAR, a failed total talus replacement, a failed ankle fusion, or an AVN talus.

In particular, the prosthetic talus described herein provides a modular total talus that allows for a defined section of the total talus to be disconnected from a base of an implant. One example would allow an articulating component to be removable which would allow a surgeon to replace just a proximal component if further articular damage occurred on the tibia. This would also allow the surgeon to convert a total talus replacement to a total ankle replacement by swapping the proximal component for a new articulating component that matched the mating geometry of a poly insert of varying total ankle implants.

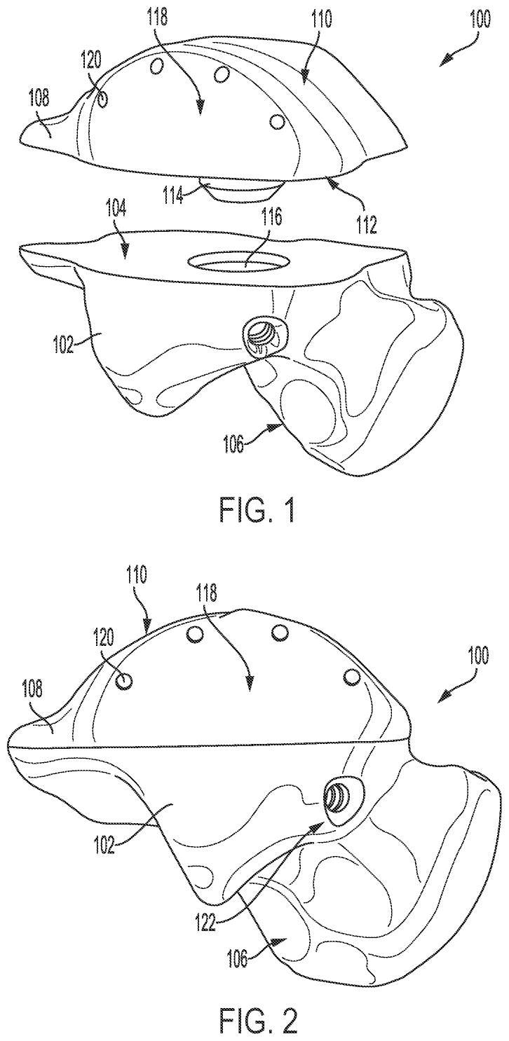

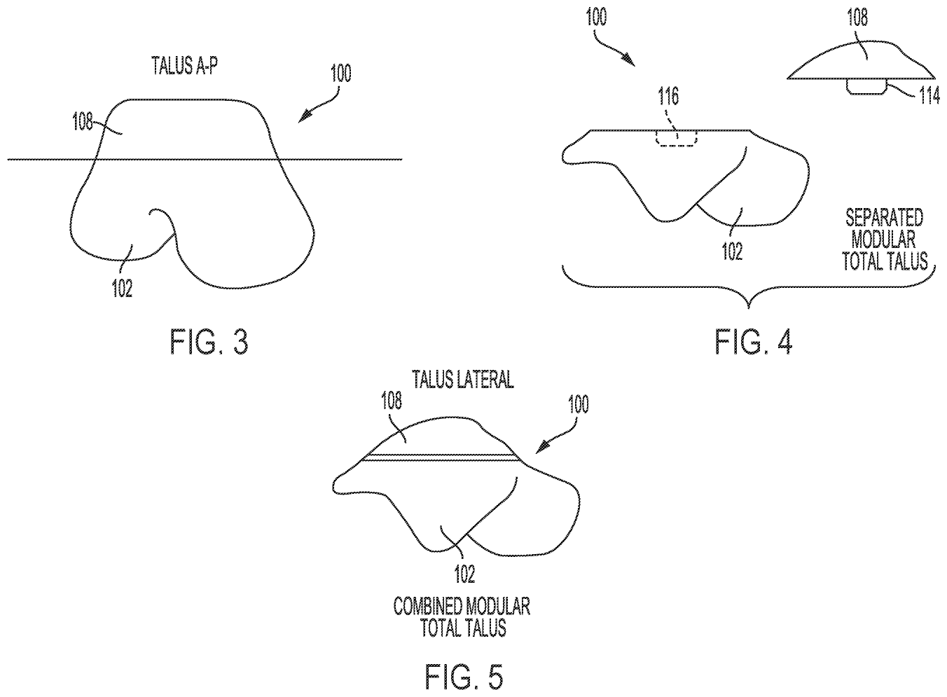

With reference to the Figures, FIGS. 1-7 illustrate a prosthetic talus 100 of a prosthetic ankle. In particular, the prosthetic talus 100 shown in FIGS. 1-7 comprises an example of a prosthetic talus 100 of a computed tomography (CT) defined patient specific talus. The prosthetic talus 100 includes a base 102 having a top surface 104 and a bottom surface 106. The prosthetic talus 100 further includes an articulating component 108 having a top surface 110 and a bottom surface 112. The bottom surface 112 of the articulating component 108 is removably coupled to the top surface 104 of the base 102. In one example, the bottom surface 112 of the articulating component 108 is removably coupled to the top surface 104 of the base 102 prior to positioning of the prosthetic talus 100 in the patient. In another example, the bottom surface 112 of the articulating component 108 is removably coupled to the top surface 104 of the base 102 in situ.

In use, the top surface 110 of the articulating component 108 is positioned adjacent a tibia of a patient, and the bottom surface 106 of the base 102 is positioned adjacent the calcaneus of the patient. The bottom surface 106 of the base 102 is also articulated against the navicular bone. In an example, the bearing surface comprises ultra-high-molecular-weight polyethylene (UHMWPE). The base 102 and the articulating component 108 of the prosthetic talus 100, when removably coupled, are shaped similar to the anatomy of the patient's talus bone. The modular aspect of the prosthetic talus 100 described herein allows the surgeon to replace just the proximal portion of the implant so it can either fit the new eroded anatomy of the distal tibia or match the mating geometry of a poly insert in a total ankle replacement implant.

In an example, the base 102 comprises a first material, and the articulating component 108 comprises a second material that is different than the first material. In one such example, the first material comprises a titanium alloy, and the second material comprises a cobalt-chromium (CoCr) alloy. Other combinations of materials are possible as well.

In an example, the bottom surface 112 of the articulating component 108 includes a protrusion 114, and the top surface 104 of the base 102 includes a recess 116 configured to receive the protrusion 114 to thereby removably couple the articulating component 108 to the base 102. In one such example, as shown in FIG. 1, the protrusion 114 and the recess 116 comprise a morse taper connection. Such an arrangement would allow for correction of internal and external rotation. In another example, the protrusion 114 and the recess 116 comprise a mechanical fastener such as a screw. In yet another example, the bottom surface 112 of the articulating component 108 includes the recess 116, and the top surface 104 of the base 102 includes the protrusion 114 configured to receive the recess 116 to thereby removably couple the articulating component 108 to the base 102.

In another example, the recess 116 comprises a feature to allow for anteroposterior positioning of the articulating component 108 with respect to the base 102. In one example, the feature comprises a channel. In another example, the feature comprises a rail. In another example, the feature comprises a plurality of holes that are evenly space. This degree of modularity would allow the mechanical axis of the tibia to coincide with weight bearing axis of the prosthetic talus 100. The articulating component 108 may be locked into final position via a fastener to thereby lock an anteroposterior position of the articulating component 108 with respect to the base 102.

As shown in FIGS. 1-2, the articulating component 108 includes a sidewall 118 positioned between the top surface 110 and the bottom surface 112. In an example, as shown in FIGS. 1-2, the sidewall 118 includes a plurality of holes 120. In another example, the plurality of holes 120 may be positioned on other surfaces of the articulating component 108. In one example, the plurality of holes 120 are configured to receive a corresponding plurality of dermal or synthetic allografts. Such a synthetic allograft may comprise silicate materials, as a non-limiting example. The articulating component 108 shown in FIGS. 1-2 may be configured to contact the ankle joint, the talonavicular joint, and the subtalar joint. Each of these joints could include a plurality of holes 120 to receive a dermal or synthetic allograft to allow for better articulation or even decompression. Such dermal or synthetic allografts may be used to lock sutures or place suture anchors to hold the allografts in place.

Traditional total talus implants may result in surrounding cartilage damage due to the articulating component 108 of the prosthetic talus 100 directly mating with the cartilage surface of the calcaneus, navicular, and tibia bones. Providing fixation methods for allografts could delay or inhibit the damage caused by the metal articulating component. In particular, the allografts act as a buffer between the articulating component 108 of the prosthetic talus 100 and the cartilage of the distal tibia, calcaneus, and/or navicular and could prolong the prosthetic talus 100 life resulting in a longer duration between revision surgeries. Further, the plurality of holes 120 shown in FIGS. 1-2 and described above would also allow for allograft to be replaced if there was minimal damage to the articulating component of the distal tibia, further prolonging the need to replace the entire total talus implant or converting the patient to a total ankle replacement.

In an example, the articulating component 108 and/or the base 102 can include suture eyelets and/or drilled and tapped holes 122 configured to accept bone anchors for lateral ankle or deltoid instability. Such suture eyelets and/or drilled and tapped holes allow the surgeon to reattach the surrounding ankle ligaments to the prosthetic talus 100 during surgery. The advantage of including tapped holes 122 to accept bone anchors would allow the procedure to utilize knotless anchor technology. The locations for the suture eyelets or tapped holes 122 for anchors may be established during a preoperative plan following a computed tomography (CT) scan.

In an example, the base 102 includes one or more holes designed to allow for subtalar, ankle fusion, TTC fusion, and/or talonavicular fusion. These holes could be located on the neck of the base 102 or underneath the neck of the base 102 to allow for screw targeting from the calcaneus of the patient. In one example, a targeting guide may be required that goes around the ankle joint. The base 102 of the prosthetic talus 100 may include features to allow for the mechanical attachment of such a targeting guide. In the case of a TTC fusion, the base 102 may include a hole to allow for insertion of a TTC nail from the subtalar joint into the intramedullary canal of the tibia of the patient.

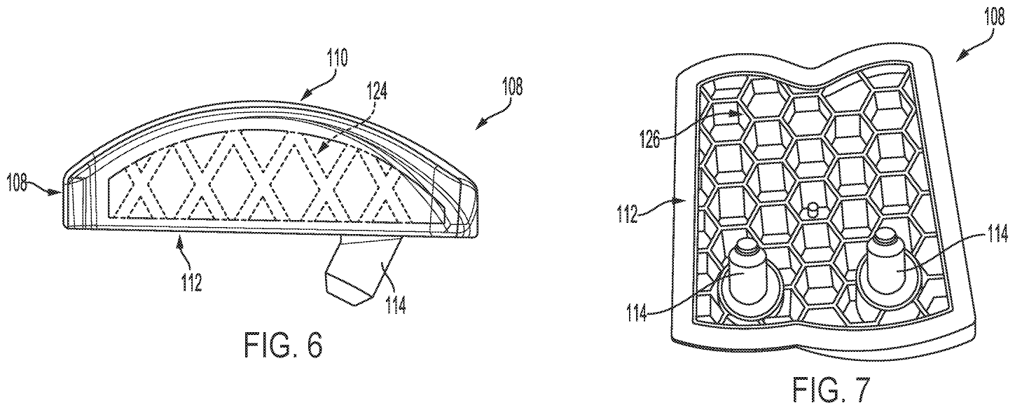

Most modern arthroplasty devices that articulate with a bearing surface are manufactured from cobalt-chromium (CoCr) alloys, ceramic alloys, oxidized Zirconium, and Nitride coated Titanium alloys for improved wear resistance. However, CoCr and the previously mentioned materials are dense materials, whose increased weight can cause increased wear against the less dense bone that the implant resides upon. In order to minimize such wear and reduce the weight of the implant, while preserving the desirable properties of CoCr, weight reducing mechanisms are desirable. In an example, as shown in FIG. 6, an interior of at least a portion of the prosthetic talus 100 is hollow. In one such example, the interior of the prosthetic talus 100 includes a lattice structure 124. Although FIG. 6 only illustrates the articulating component 108 with the lattice structure 124, the base 102 may include a similar lattice structure 124. In an example, an entirety of the interior of the base 102 and/or an entirety of the interior of the articulating component 108 comprises the lattice structure 124. In another example, the interior of base 102 and/or the interior of the articulating component 108 includes alternating solid layers and lattice structure layers. The solid and lattice layers can be manufactured from the same material (such as CoCr) or a variation of mixed material layers. This same material may also comprise the shell of the base 102 and/or the shell of the articulating component 108 as well. The lattice structure 124 positioned in the hollow interior of the prosthetic talus 100 that adds strength to the implant can be either be a uniform beam design or a formula driven gyroid shape.

In an example, as shown in FIG. 7, an interior of at least a portion of the prosthetic talus 100 is hollow. In one such example, the interior of the prosthetic talus 100 includes a honeycomb structure 126. Although FIG. 7 only illustrates the articulating component 108 with the honeycomb structure 126, the base 102 may include a similar honeycomb structure 126. The honeycomb structure 126 may provide additional strength to the prosthetic talus 100 while reducing weight. Further, the honeycomb structure 126 may provide an addition structure through which the lattice structure 124 may be positioned.

As further shown in FIGS. 6-7 and as discussed above, the articulating component 108 includes a protrusion 114, and the top surface 104 of the base 102 includes a recess 116 configured to receive the protrusion 114 to thereby removably couple the articulating component 108 to the base 102. In an example, as shown in FIG. 6, the protrusion 114 comprises a single protrusion. In another example, as shown in FIG. 7, the protrusion 114 comprises a pair of protrusions. In an example, an interior of the protrusion 114 is solid. In another example, an interior of the protrusion 114 is hollow and includes a lattice structure similar to the lattice structure 124 of the main body of the prosthetic talus 100 discussed above. In an example, the protrusion 114 is angled between 0 and 90 degrees with respect to the bottom surface 112 of the articulating component 108. In another example, the protrusion 114 is perpendicular to the bottom surface 112 of the articulating component 108.

In addition to the advantages described above, the prosthetic talus 100 described above in relation to FIGS. 1-7 provides a benefit in the surgical approach. A traditional total talus can be very large and at times a surgeon may have to break the fibula for access and then reattach the fibula after installation of the total talus. Since the prosthetic talus 100 described above includes a bottom surface 112 of the articulating component 108 that is removably coupled to a top surface 104 of the base 102, a surgeon can assemble the prosthetic talus 100 in two components. This enables a surgeon to perform the procedure via an anterior approach, which is the standard approach for doing a total ankle replacement surgery. Further, separating the prosthetic talus 100 into two components may allow the prosthetic talus 100 to have better fixation for bone screws which will help anchor the prosthetic talus 100 to the calcaneus.

In an example, one or more components of the prosthetic talus 100 described above in relation to FIGS. 1-7 are configured to be positioned in contact with a bone of a patient, and at least a portion of an exterior surface of the prosthetic talus 100 includes a Zinc-Strontium (Zn—Sr) alloy. In such an example, the Zn—Sr alloy is selected from the group consisting of Zn—Sr, Zn-0.8Sr, Zn-0.6 Sr, Zn-0.5Sr, Zn-0.4Sr, Zn-0.2Sr, and Zn-0.1 Sr. Once the prosthetic talus 100 is in contact with the bone of the patient, the Zn—Sr alloy stimulates osteogeneis of mesenchymal stem cells at the implant site. In an example, the Zn—Sr alloy stimulates mesenchymal stem cells selected from the group consisting of CD45−, CD45−CD146+, CD45-CD271+, CD31-44+45-73+90+105+, and CD45-CD34+. Further, the Zn—Sr alloy increases cellular PI3K/Akt, MAPK/Erk, and/or Wnt/B-catenin pathway signaling, thereby promoting anabolic and anticatabolic effects on bone remodeling. In an example, the Zn—Sr alloy further includes a material selected from the group consisting of tricalcium phosphate (TCP), hydroxyapatite (HA), and Silicon. In another example, the Zn—Sr alloy includes no more than a trace amount of Magnesium.

The addition of Sr—Zn based metals to one or more components of the prosthetic talus 100 would help promote and/or stimulate new bone formation while also inhibiting bone resorption during the healing process, thereby reducing potential failure modes associated with implant loosening and subsidence.

In an example, the Zn—Sr alloy comprises a three-dimensional structure extending away from the exterior surface of the prosthetic talus 100. In one such example, the three-dimensional structure comprises a scaffold.

In some examples, such as shown in any one of FIGS. 1-7, one or more components of the prosthetic talus 100 is made via an additive manufacturing process using an additive-manufacturing machine, such as stereolithography, multi-jet modeling, inkjet printing, selective laser sintering/melting (or DMLS, EBM), and fused filament fabrication, among other possibilities. Additive manufacturing enables one or more components of the prosthetic talus 100 and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the prosthetic talus 100 using a Computer Aided Design (CAD) of the prosthetic talus 100 as instructions. As a result, changes to the design of the prosthetic talus 100 can be immediately carried out in subsequent physical creations of the prosthetic talus 100. This enables the components of the prosthetic talus 100 to be easily adjusted or scaled to fit different types of applications (e.g., for use with various types and sizes of prosthetic ankles).

The layer-upon-layer process utilized in additive manufacturing can deposit one or more components of the prosthetic talus 100 with complex designs that might not be possible for devices assembled with subtractive manufacturing. In turn, the design of the prosthetic talus 100 can include aspects that aim to improve overall operation. For example, the design can incorporate physical elements that help redirect stresses in a desired manner that traditionally manufactured devices might not be able to replicate.

Additive manufacturing also enables depositing one or more components of the prosthetic talus 100 in a variety of materials using a multi-material additive-manufacturing process. In such an example, as discussed above, the base 102 may be made from a first material, and the articulating component 108 may be made from a second material that is different than the first material. In another example, the entire prosthetic talus 100 is made from the same material. Other example material combinations are possible as well. Further, one or more components of the prosthetic talus 100 can have some layers that are created using a first type of material and other layers that are created using a second type of material.

In an example, an interior of one or more components the prosthetic talus 100 is hollow. In one such example, the interior of the base 102 and/or articulating component 108 includes a lattice structure. In an example, an entirety of the interior of the base 102 and/or the articulating component 108 comprises the lattice structure. In another example, the interior of the base 102 and/or the base 102 and/or the articulating component 108 includes alternating solid layers and lattice structure layers. The solid and lattice layers can be manufactured from the same material (such as CoCr) or a variation of mixed material layers. This same material may also comprise the shell of the base 102 and/or the articulating component 108 as well. The lattice structure positioned in the hollow interior of the base 102 and/or the articulating component 108 that adds strength to the implant can be either be a uniform beam design or a formula driven gyroid shape.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” or “an example” means that one or more feature, structure, or characteristic described in connection with the example is included in at least one implementation. The phrases “one embodiment” or “one example” or “an example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ±5% of a given value.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

The limitations of the following claims are not written in means-plus-function format and are not intended to be interpreted based on 35 U.S.C. § 112 (f), unless and until such claim limitations expressly use the phrase “means for” followed by a statement of function void of further structure.

1. A prosthetic talus comprising:
a base having a top surface and a bottom surface; and
an articulating component having a top surface and a bottom surface, wherein the bottom surface of the articulating component is removably coupled to the top surface of the base, wherein the bottom surface of the articulating component includes a protrusion, and wherein the top surface of the base includes a recess configured to receive the protrusion to thereby removably couple the articulating component to the base, wherein the articulating component includes a sidewall positioned between the top surface and the bottom surface, and wherein the sidewall includes a plurality of holes.

2. A prosthetic talus comprising:
a base having a top surface and a bottom surface; and
an articulating component having a top surface and a bottom surface, wherein the bottom surface of the articulating component is removably coupled to the top surface of the base.

3. The prosthetic talus of claim 2, wherein the base comprises a first material, and wherein the articulating component comprises a second material that is different than the first material.

4. The prosthetic talus of claim 3, wherein the first material comprises a titanium alloy, and wherein the second material comprises a cobalt-chromium (CoCr) alloy.

5. The prosthetic talus of claim 2, wherein the bottom surface of the articulating component includes a protrusion, and wherein the top surface of the base includes a recess configured to receive the protrusion to thereby removably couple the articulating component to the base.

6. The prosthetic talus of claim 5, wherein the protrusion and the recess comprise a morse taper connection.

7. The prosthetic talus of claim 5, wherein the recess comprises a feature to allow for anteroposterior positioning of the articulating component with respect to the base.

8. The prosthetic talus of claim 7, further comprising a fastener configured to lock an anteroposterior position of the articulating component with respect to the base.

9. The prosthetic talus of claim 2, wherein the articulating component includes a sidewall positioned between the top surface and the bottom surface, and wherein the sidewall includes a plurality of holes.

10. The prosthetic talus of claim 9, wherein the plurality of holes are configured to receive a corresponding plurality of dermal or synthetic allografts.

11. The prosthetic talus of claim 2, wherein the articulating component and/or the base include suture eyelets and/or drilled and tapped holes configured to accept bone anchors for lateral ankle or deltoid instability.

12. The prosthetic talus of claim 2, wherein the base includes one or more holes designed to allow for subtalar/ankle fusion or talonavicular fusion.

13. The prosthetic talus of claim 12, wherein the one or more holes are located on a neck of the base to allow for screw targeting from a calcaneus of a patient.

14. The prosthetic talus of claim 2, wherein at least a portion of an exterior surface of the bottom surface of the base includes a Zinc-Strontium (Zn—Sr) alloy and/or wherein at least a portion of an exterior surface of the top surface of the articulating component includes a Zn—Sr alloy.

15. The prosthetic talus of claim 14, wherein the Zn—Sr alloy is selected from the group consisting of Zn—Sr, Zn-0.8Sr, Zn-0.6 Sr, Zn-0.5Sr, Zn-0.4Sr, Zn-0.2Sr, and Zn-0.1 Sr.

16. The prosthetic talus of claim 14, wherein the Zn—Sr alloy stimulates osteogeneis of mesenchymal stem cells selected from the group consisting of CD45−, CD45−CD146+, CD45−CD271+, CD31−44+45−73+90+105+, and CD45−CD34+.

17. The prosthetic talus of claim 14, wherein the Zn—Sr alloy further includes a material selected from the group consisting of tricalcium phosphate (TCP), hydroxyapatite (HA), and Silicon.

18. The prosthetic talus of claim 14, wherein the Zn—Sr alloy includes no more than a trace amount of Magnesium.

19. The prosthetic talus of claim 14, wherein the Zn—Sr alloy comprises a three-dimensional structure extending away from the exterior surface of the bottom surface of the base and/or the Zn—Sr alloy comprises a three-dimensional structure extending away from the exterior surface of the top surface of the articulating component.

20. The prosthetic talus of claim 19, wherein the three-dimensional structure comprises a scaffold.
